(19) United States
(12) Patent Application Publication
(10) Pub. No.: US 2013/0040109 A1
(43) Pub. Date: Feb. 14, 2013

(54) BIODEGRADABLE MEDICAL MATERIAL

(71) Applicants: Laurent Lebrette, Tresserre (FR); Jean-Christophe Ricatte, Amelie Les Bains (FR); Christophe Simon, Maureillas Las Illas (FR)

(72) Inventors: Laurent Lebrette, Tresserre (FR); Jean-Christophe Ricatte, Amelie Les Bains (FR); Christophe Simon, Maureillas Las Illas (FR)

(73) Assignee: ARJOWIGGINS HEALTHCARE, Amelie Les Bains Palalda (FR)

(21) Appl. No.: 13/634,137

(22) PCT Filed: Mar. 11, 2011
(86) PCT No.: PCT/IB2011/051039

§ 371 Date: Oct. 19, 2012

Related U.S. Application Data
(63) Continuation-in-part of application No. 12/721,632, filed on Mar. 11, 2010.

(51) Int. Cl. D21H 11/00; D21H 11/12; A41D 13/12; B32B 29/06; D04H 3/04; A61B 19/08

(52) U.S. Cl. 428/153; 28/100; 128/849; 128/853; 2/114; 428/156; 428/219; 162/141; 162/148

(57) ABSTRACT

A biodegradable monolayer medical material, comprising at least 15 percent cellulose fibers by dry weight, with a remainder comprising at least 60 percent by dry weight of natural fibers, the monolayer medical material providing a barrier to liquids and/or microorganisms meeting at least level 1 performance based on a standard defined by AAMI PB70, and at least a portion of the monolayer medical material comprises a softening process pattern configured to soften the monolayer medical barrier material.

### FIELD OF THE DISCLOSURE

The present disclosure relates to paper products, and more particularly to biodegradable medical barrier materials.

### BACKGROUND OF THE DISCLOSURE

Medical barrier products have seen increased use throughout the world and contribute to human generated waste. Among these medical barriers are drapes, sterile barriers, sterilization wraps, gowns, etc., which have grown in use as more individuals throughout the world seek medical care, and medical centers (e.g., hospitals, clinics, etc.) become more prevalent. Such medical barriers may be used during any number of procedures, from a chair or bench covering in a general practitioner's office, to a sterile barrier in an operating room (“OR”), to a wrap for maintaining sterile conditions for surgical tools, to a surgical gown worn by an attending physician.

Because of a desire to maintain certain levels of barrier performance while also providing a strong, safe, comfortable, and easy to shape material, medical barrier materials are often comprised of fluorinated, multi-layer, woven and non-woven fabrics such as spunbond and meltblown (e.g., SMS) fibers utilizing, for example, PET, polyolefins such as polypropylene, and polyethylene, polyamides, and/or other synthetic compounds. However, these products may not provide a desirable level of biodegradability and some may have properties compromised by, for example, hand lotions that may be present on the hands of various personnel working with the barriers. This may be particularly true with regard to water and/or alcohol repellency of such barriers.

In addition, other medical barrier materials may be formed of multiple and varying layers of material. For example, a first layer may comprise a non-woven material, while a second layer may include a plastic film or similar.

These two layers may then be bonded by additional—and potentially costly—offline manufacturing steps (e.g., gluing, laminating, etc.) together to result in a finished sheet.

Many medical barrier materials, both synthetic and non-synthetic may include various substances which have been classified as harmful to the environment (i.e., eco-toxic) for enhancing various characteristics (e.g., barrier performance, feel, tear strength, conformability, etc.). These substances may include, for example, fluorine, arsenic, selenium, molybdenum, chromium, mercury, lead, cadmium, nickel, copper, zinc, etc.

Because of sanitary concerns, among other things, medical barrier materials are generally not reusable and are typically disposed of via various methods (e.g., landfill and/or incineration) thereby potentially contributing to harmful environmental effects. For example, substances classified as harmful to the environment may escape the paper products during disposal and be transmitted directly to the surrounding soil and/or atmosphere. The environmental effects of these compounds may increase as their concentration increases, and it is therefore desirable to limit and/or eliminate the possibility of such compounds escaping by minimizing or eliminating their use in the barrier products.

Moreover, depending on application (e.g., surgical drape set), barrier products may consume relatively large amounts of space in landfills and/or other trash locations, and may have unacceptably long degradation times under natural circumstances (e.g., hundreds or thousands of years). Therefore, it is desirable to produce a medical barrier material having improved degradation properties, while still providing desirable functional characteristics (e.g. barrier performance, tear strength, conformability, and feel).

Until now, utilization of non-woven fabrics from natural fibers (e.g., cellulose) as medical barrier materials may provide some desirable characteristics related to biodegradability, but may suffer from drawbacks such as poor conformability, poor barrier performance, an undesirable feel (e.g., coarse), and undesirable breathability, among other things. Additionally, characteristics of articles formed with natural fibers may vary widely based on the quality and size of the fibers used to form the article.

U.S. Patent Publication No. 2007/0238384 describes biodegradable and/or water soluble laminate articles and operating room drapes. The articles and operating room drapes are described as including at least a first layer of non-woven fabric, a second layer of non-woven fabric, and a third layer of a polymeric film positioned and bonded between the first and second layer. Such an arrangement may add costs during manufacturing based at least on the additional materials and bonding steps involved.

U.S. Pat. No. 4,622,259 describes a non-reinforced microfiber fabric having particular characteristics for use in medical applications. The fabric is made by thermally embossing a microfiber web comprising a polymer in a melt-blown type process. Such melt-blown polymer webs may present issues such as slow biodegradation, among other things.

U.S. Pat. No. 5,180,614 describes supple sheets including cellulose fibers and optionally synthetic fibers, and a binder. The sheets can include a moisture retaining agent and may be heat sealed with plastic films. Such configurations may also involve additional materials and added costs. In addition, undesirable compounds may be introduced to the environment when such articles are disposed of.

U.S. Pat. No. 5,560,974 describes a breathable non-woven composite fabric bonded to a surface of a thermoplastic microporous film. Such designs may increase costs for at least the reason that additional fabrication steps (e.g., gluing, bonding, laminating) are utilized following preparation of the non-woven material.

U.S. Pat. No. 5,783,504 describes plies of nonwoven bonded with plies of a thermoplastic film into biodegradable structures, the structures having applications in the medical field. The non-woven plies are totally manufactured from a polymer or a copolymer or a blend polymers derived from lactic acid, while the film plies are manufactured totally from a polymer of biodegradable aliphatic polyester. Such a configuration may involve a bonding step for each of the plies involved, thereby increasing time and cost during manufacture.

PCT Patent Application WO 97/31153 describes a process for softening cellulose fibers and articles made therefrom (e.g., non-woven fabrics). The process includes treating the cellulose fiber with chemical softening compositions comprising phospholipids, non-ionic surfactants, and lubricating agents. Based at least on the addition of the chemical softening compositions, such a configuration may lead to undesirable levels of chemical compounds in the environment, and may further add cost to the manufacturing process.

It is accordingly desirable to provide a medical barrier material presenting a desirable combination of conformability, biodegradability, and barrier performance (e.g., liquid and microorganism barrier), while reducing and/or eliminating the inclusion of certain environmentally harmful elements. More particularly, medical barrier materials that are substantially biodegradable and non-ecotoxic are desired. The medical barrier materials disclosed herein maintain airborne and liquid and microorganism barrier to at least levels 1, 2, and 3 according to the Association for the Advancement of Medical Instrumentation (AAMI) PB70 (2008 version) standards, and provide desirable breathability and conformability of the material.

### SUMMARY OF THE DISCLOSURE

In accordance with the present disclosure, a biodegradable monolayer medical material is provided. The medical material includes at least 15 percent cellulose fibers (for example, bleached, semi bleached, and/or unbleached) by dry weight, with a remainder consisting of at least 60 percent by dry weight (with respect to the total dry weight of the medical material) of natural fibers. The monolayer medical material provides a barrier to liquids and/or microorganisms meeting at least level 1 performance based on AAMI PB70 standard and at least a portion of the monolayer medical material comprises a softening process pattern configured to soften the monolayer medical barrier material.

Based on such a configuration, it may be possible to provide an environmentally friendly medical material providing desirable performance characteristics. Such a material may limit and/or eliminate off-wire processing (e.g., gluing, laminating, etc.) and may further limit and/or eliminate eco-toxic compounds from such materials. Moreover, such configurations enable a wide range of natural fibers to be utilized, while still maintaining desired biodegradability.

The biodegradable monolayer medical material can be configured to meet at least level 2 performance requirements based on the standard defined by AAMI PB70.

The biodegradable monolayer medical material can be configured to meet at least level 3 performance requirements based on the standard defined by AAMI PB70.

The softening process pattern can be selected from one of a creped pattern, a micro-creped pattern, and an embossing, and may be performed on-line and/or off-line. As used herein the term “on-line” shall mean a period during which the material is formed (e.g., on the wire), and includes any process from wet laying through finishing (e.g., drying, calendaring, sizing, etc.). The term “off-line” shall mean any period after the on-line period.

The softening process pattern can result in a machine direction drape value less than 200 mm, and according to some embodiments less than 120 mm, for example between about 25 and 100 mm.

According to some embodiments, the cellulose fiber may be bleached, semi bleached, and/or unbleached cellulose, and may be virgin or recycled. The natural fibers can be selected from at least one of bleached wood pulp, semi-bleached wood pulp, unbleached wood pulp. The natural fibers comprising the remainder may be selected from any of unbleached cellulose, cotton fibers, abaca (e.g., from Ogura Trading Co. Ltd. for instance), straw (e.g., rice straw fibers from Ogura Trading), bamboo (e.g., from OG Corporation), hemp, jute, sisal, asperto (alfa), eucalyptus, and/or viscose fibers (e.g., TENCEL LENZING VISCOSE fibers, and/or various fibers produced by Kelheim Fibres GmbH such as, for example DANUFIL®, VILOFT®, GALAXY®, BRAMANTE, DANTE, BELLINI, etc.)Such natural fibers of the remainder inherently contain additional cellulose fibers (in addition to other compounds such as lignin, pectin, hemicelluloses, etc.), which is not counted with regard to the at least 15 percent cellulose fibers. The biodegradable monolayer medical material may include less than less than 20 percent microcrystalline cellulose, and in some embodiments, less than 10 percent microcrystalline cellulose, for example 5 percent microcrystalline cellulose.

The biodegradable monolayer medical material can include less than 20 percent by dry weight of at least one of synthetic compound, a compound with unknown biodegradability, and a non-biodegradable compound. The synthetic compound and/or the non-biodegradable compound can be as for an example an acrylic, styrene butadiene, vinyl acetate, or preferably styrene acrylic emulsions.

The biodegradable monolayer medical material can have a relative aerobic biodegradability of at least 80 percent after 180 days, and in some embodiments, greater than 90 percent after 40 days.

The biodegradable monolayer medical material can be void of any coloring agent, neither on a surface of the material nor within the structure.

The biodegradable monolayer medical material may be void of any eco-toxic substances in a concentration greater than 200 parts per million. According to some embodiments, the biodegradable monolayer medical material may be void or substantially void of perfluoro octanoic acid (PFOA).

The biodegradable monolayer medical material may have a grammage between 25g/m² and 200 g/m². In some embodiments, this grammage may be before finishing (e.g., softening process).

The biodegradable monolayer medical material may have a linting value of less than 10 and according to some embodiments of the present disclosure, less than 5.0.

Further, the cellulose fibers of the biodegradable monolayer medical material may have an average length of between about 0.1 millimeter to 30 millimeters and according to some embodiments of the present disclosure, from 1 to 15 millimeters.

A sterilized surgical drape may be fabricated from the biodegradable monolayer medical material described herein. The surgical drape may have a hole provided in a predetermined location.

A sterilized gown may be fabricated from the biodegradable monolayer medical material described herein.

A sterilized sterilization package may be fabricated from the biodegradable monolayer medical material described herein.

According to some embodiments, the biodegradable monolayer medical material may be packed in a sterilized sterile barrier system as predefined by ISO 11607 (version of August 2009) or used as a component of a sterile barrier system as defined by ISO 11607.

According to another embodiment of the present disclosure, a method for manufacturing a biodegradable monolayer medical material is provided. The method includes preparing a furnish comprising at least 15 percent cellulose fibers by dry weight, with a remainder consisting of at least 60 percent by dry weight of natural fibers, forming a sheet from the furnish in a wet-laid process, drying the sheet,and forming a pattern on and/or within the sheet via a softening process configured to result in a softening of the sheet, the sheet having a barrier performance meeting at least level 1 according to the AAMI PB70 standard.

The softening process can be selected from a, creping, micro-creping and an embossing, and may be performed either offline and/or on-line.

The softening process may be a mechanical process and may be configured to result in a machine direction drape value less than 200 mm and, according to some embodiments of the present disclosure, less than 120 mm. The softening process may include at least one of a creping process, a microcreping process, and an embossing process.

The drying can be carried out a temperature between about 75 degrees C. and about 200 degrees C. The wet-laid process may be selected from one of a fourdrinier process, an inclined wire process, and a mold table process.

Additional objects and advantages of the present disclosure will be set forth in part in the description which follows.

It is to be understood that both the foregoing general description and the following detailed description are exemplary and explanatory only and are not restrictive of the present disclosure, as claimed.

### BRIEF DESCRIPTION OF THE DRAWINGS

The accompanying drawings, which are incorporated in and constitute a part of this specification, illustrate a number of exemplary features of non-limiting embodiments of the disclosure and together with the description, serve to explain the principles of the disclosure. In the drawings,

FIG. 1A is an exemplary illustration of an arrangement for a set of operating room drapes;

FIG. 1B is an exemplary illustration of a sterile field or Mayo table cover.

FIG. 2 is a cross section of an exemplary medical barrier material consistent with embodiments of the present disclosure.

### MORE DETAILED DESCRIPTION

Reference will now be made in detail to the exemplary embodiments of the present disclosure, examples of which are provided and illustrated in the accompanying drawings. Wherever possible, the same reference numbers will be used throughout the drawings to refer to the same or like parts.

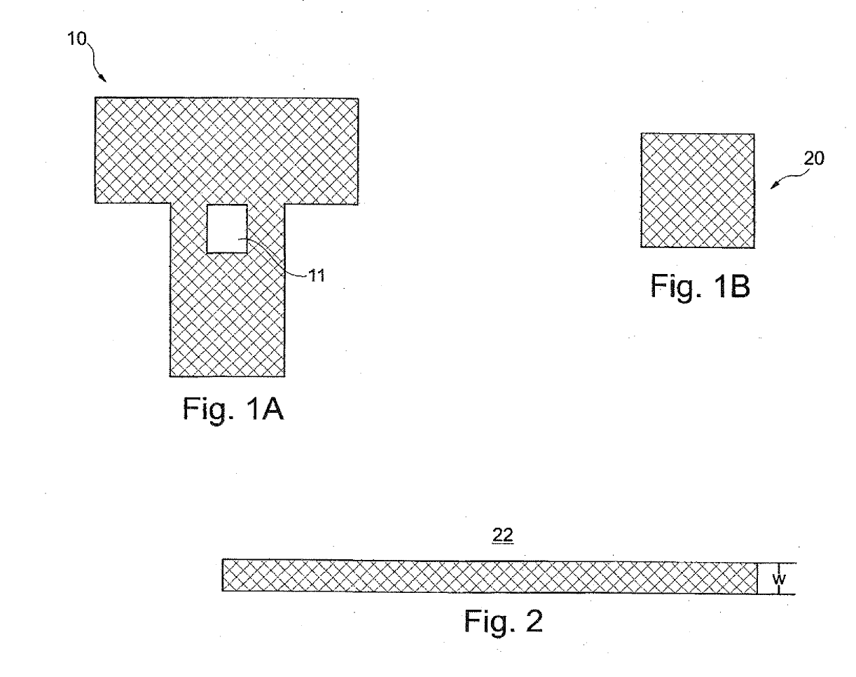

FIG. 1A is an exemplary illustration of an arrangement for a set of operating room drapes 10 while FIG. 1B is an exemplary sterile field or Mayo table cover 20. Many operating rooms include a table or platform of some variety for supporting a patient, as well as various equipment trays and/or supply trays configured to support and provide access to various implements for performing surgical procedures. In many situations, it may be desirable to provide a barrier not only on top of such supports, but also a cover to protect portions of the patient and gowns to protect portions of the physicians. For example, where a surgical drape is used a covering for a patient, an opening 11 may be provided in the drape at an access location (e.g., where a procedure is to be carried out on the patient's body) among other things. In some embodiments, surgical drapes may include other materials and/or features (e.g., pleats) to enhance for example, strength and/or liquid repellency in desired locations of the material.

Notably, the present disclosure is not intended to be limiting, and embodiments of the present disclosure have application outside of surgical drapes and implements within an operating room. For example, embodiments of the present disclosure may have application to gowns, sterile fields, sterile barrier system, facial masks, head coverings, and surgical drapes, among others. FIGS. 1A and 1B are intended to be exemplary only.

FIG. 2 is a cross section of an exemplary medical barrier material 22 consistent with embodiments of the present disclosure. Embodiments of the present disclosure utilize a single layer (i.e., mono-layer) non-woven sheet 22 having a thickness W, formed from a furnish comprising natural fibers, for example, selected from bleached wood pulp, semi-bleached wood pulp, unbleached wood pulp, cotton fibers, abaca, straw, bamboo, hemp, jute, sisal, esparto (alfa) and viscose, among others. According to some embodiments, a furnish containing at least 15 percent bleached cellulose fibers by dry weight for the total composition of fibers, the remainder of the fibers including other natural fibers may be utilized for manufacturing sheets of the medical barrier material. For example, in addition to 20 percent bleached cellulose fibers, 80 percent unbleached cellulose fibers by dry weight may be included in the composition. In some embodiments, up to 40 percent bleached cellulose fibers may be used in combination with 60 percent unbleached cellulose fibers, for example.

In embodiments of the present disclosure implemented with cellulose, any suitable pulps may be utilized for obtaining the cellulose, for example, kraft pulps from coniferous and/or deciduous trees. Portions of the cellulose may be mechanically, chemically, thermo-mechanically, and/or chemi-thermo-mechanically pulped, as desired. Moreover, where desired, microcrystalline cellulose may be utilized as a portion of the cellulose fibers. This may be desirable in some embodiments, for example, to enhance biodegradability further of sheet 22.

Fiber lengths associated with the various natural fibers may be, for example, between about 0.1 millimeter to about 30 millimeters, and in some embodiments may be from 1 to 15 millimeters.

Sheet 22 may be produced on an inclined wire device, Fourdrinier paper machine (“the wire”), mold, or any other suitable device for performing a wet-laid process. For example, bleached and unbleached cellulose fibers may be mixed into suspension in aqueous medium (i.e., the furnish) in proportions of 15 to 25 percent, for example, 20 percent, and 75 to 85 percent, for example, 80 percent respectively by dry weight. This suspension can be dewatered on the wire of the machine in order to form the sheet. The resulting sheet may be have a grammage in a range of, for example, 50 to 150 grams per square meter (g/m²), from 60 to 90 g/m², or 70 to 85 g/m².

Thicknesses associated with sheet 22 following formation may range from, for example, about 90 micrometers to about 1000 micrometers.

In some embodiments a binder, and/or a non biodegradable compound, and/or a compound whose biodegradability is unknown may be utilized during the formation process, for example, polyvinyl alcohol, a starch, and/or a polymer added in the form of a stable aqueous emulsion, especially acrylic polymers or acrylates (vinyl acrylic-ester copolymer) or styrene butadiene rubber or acrylic-styrene polymers. Such compounds may be added in an amount ranging from 0 percent to 20 percent by dry weight, and according to some embodiments, less than 5 percent. For example, a binder may be added at 2 percent by dry weight with the natural fibers. Such addition may be made in the furnish (i.e., the aqueous solution) and/or following formation on the wire to sheet 22.

Further, according to some embodiments, a coloring agent (e.g., a pigment and/or a dye) may be introduced into the aqueous solution where desired and/or sheet 22 may be printed or otherwise colored following formation on the wire. For example, where a color match is desired, an appropriate pigment and/or dye (e.g., a blue pigment) may be added to cause the dried sheet to have a desired color. The pigments and/or dyes may be natural and/or synthetic, and combinations thereof, and such pigments may be biodegradable. Alternatively, sheet 22 may be void of any coloring agent and may be colored based substantially on the fibers used to manufacture sheet 22 and other environmental conditions.

Following formation, sheet 22 may undergo one or more off line or/ on-line finishing processes. For example one or more softening processes (e.g., mechanical finishing) may be applied to sheet 22 as desired. Such softening processes may be configured to effect a softening of the sheet, an increase in strength, an increase in breathability, and/or an increase in conformability, among other things. Such softening processes may therefore be effective to at least reduce drape values (i.e., increase conformability).

Softening processes may include, for example, mechanical processes such as creping, micro-creping, flexage, embossing, etc. Micro-creping, for example, may act on the web associated with the sheet by compacting it, particularly in the machine direction (MD). Therefore, when exposing to a micro-crepe finishing process, it may be desirable to have a certain percentage of fibers of the web oriented in the machine direction, thereby allowing more fibers to be creped.

According to some embodiments, the finishing processes (e.g., softening) may configured to form a pattern on and/or within the structure of sheet 22 resulting in, for example, a softening process pattern on and/or within the structure of sheet 22. Such a pattern may be a visible pattern, semi-visible, or not visible to the naked eye (e.g., microscopic pattern), as desired, and/or combinations thereof. The mechanical finishing processes may be executed on any suitable processor, for example, when micro-creping sheet 22, a mechanical microcreper may be utilized. According to some embodiments, a MICREX® Microcreper may be implemented to micro-crepe the formed sheets. For example, a microcreper having rigid retarders, and/or comb roll cavity, and/or two rolls cavity, and/or flat blade cavity, or bladeless microcreper may be utilized.

According to some embodiments, coloring agents may be added to provide color to the sheet after the mechanical finishing process, particularly where coloring agents may affect the mechanical finishing process (e.g., depending on sensitivity of a creping machine). For example, a blue pigment may be introduced into the aqueous solution prior to formation on the wire. Additionally, it may be possible to provide designs and/or text, among other things, via printing on the sheet. Alternatively, no coloring agents may be used.

Medical barriers, such as surgical drapes, surgical gowns, etc. may be sterilized and provided in a sterilized packaging following their manufacture. For example, a surgical gown manufactured according to embodiments of the present disclosure

Based on the natural composition of sheet 22, sheet 22 may demonstrate desirable biodegradability, strength, barrier, conformability, and breathability properties, among others. Particularly the sheet may reach a minimum aerobic biodegradation of 90 percent and even up to 100 percent relative to cellulose after 28 days at an ambient temperature of 21 degrees C. Sheet 22 may reach an anaerobic biodegradation of 95 percent or higher after 15 days at 52 degrees C. in an activated sludge digester. In addition, sheet 22 may be low-linting.

Further, sheet 22 may be substantially void, for example, less than 200 parts per million (PPM), less than 100 PPM, or less than 50 PPM of any ecotoxic substance (e.g., fluorine, arsenic, selenium, molybdenum, chromium, mercury, lead, cadmium, nickel, copper, zinc, etc.) According to some embodiments, sheet 22 may be substantially void or completely void of perfluoro octanoic acid (PFOA), in other words, substantially or completely PFOA free.

Moreover, because the sheet is a single layer producing desired characteristics, offline manufacturing steps such as laminating, gluing, complexing, etc. monolayer embodiments of the present disclosure may be limited and/or avoided.

Analytical Testing

Sheets 22 formed according to embodiments of the present disclosure were tested according to standards set forth by various administrative standards bodies throughout the world. Testing according to some of the standards will be discussed briefly herein with regard to actual practice for the following examples.

Barrier Performance

The standard AAMI PB70 related to liquid and airborne microorganism barriers, sets forth two tests and related benchmarks to determine whether a material meets level 2 and/or level 3 standards. These tests are the spray impact test method (AATCC 42 (2007 version)) and the hydrostatic test method (AATCC 127 (2008 version)). These standards will be discussed with reference to the examples below. To meet level 2 and/or level 3 barrier requirements of these tests, the specific benchmarks of both tests must be reached, while a material meeting the benchmark for the spray impact test (i.e., 4.5 grams or less weight gain for a collecting blotter) will qualify as a level 1 barrier. These benchmarks are displayed at Table 1. While it remains possible that the AAMI may change the benchmark values associated with the described barrier levels (i.e., those associated with the PB70 standard), it is intended that for purposes of determining the scope of the present disclosure, the benchmark values and their associated levels remain as disclosed herein.

TABLE 1

| | Level 1 Performance | Level 2 Performance | Level 3 Performance |
|---|---|---|---|
| Spray Impact | less than or equal to | less than or equal to | less than or equal to |
| Test (AATCC 42) | 4.5 grams gain in mass | 1 gram gain in mass | 1 gram gain in mass |
| Hydrostatic Test | NA | resists penetration at | resists penetration at |
| (AATCC 127) | | greater than or equal | greater than or equal |
| | | to 20 cm of water | to 50 cm of water |

Biodegradability

Aerobic biodegradability was measured based on methods set forth in ISO 14851 (2009 version). Testing for aerobic biodegradability took place at an ambient temperature of approximately 21 degrees C.

Conformability

The conformability of a material is its ability to drape easily the shape of the object that it is covering. The conformability was measured according to the drape method described in EN 868-2 (1999 version-Annex. D): 2009 appendix C. It is expressed in mm.

Linting

Linting was measured according to ISO 9073-10 (June 2005) standard whose principle is the measurement of particles from 3 μm up to 10 μm, that are pulled apart from a material that is stretched and twisted within 300 seconds. It is expressed by the log value of the number of particles that has fallen down from the material during the test.

TABLE 2

| Examples | 1 | 2 | 3 | 4 | 5 |
|---|---|---|---|---|---|
| Cellulose fibers | 34% (>15%) | 21% (>15%) | 16% (>15%) | 29% (>15%) | 16 (>15%) |
| Natural fibers | 64% (>60%) | 76% (>60%) | 82% (>60%) | 69% (>60%) | 78% (>60%) |
| Microcrystalline | — | — | — | — | 5% |
| cellulose | | | | | |
| Binder and or other | 2% | 3% | 2% | 2% | 1% |
| compound with unknown | | | | | |
| biodegradability | | | | | |
| Ecotoxic compounds | Less than | Less than | Less than | Less than | Less than |
| | 200 ppm | 200 ppm | 200 ppm | 200 ppm | 200 ppm |
| PFOA | free | free | free | free | free |
| Softening process | on-line | off-line | off-line | off-line | off-line |
| Softening | Creped | micro-creped | micro-creped | micro-creped | micro-creped |
| Technique | | | | | |

As shown in Table 2, in the invention, the total amount (to 100 percent) is the addition of at least 15 percent cellulose fibers and of the remainder comprising natural fibers that amount to at least 60 percent of the total amount.

### EXAMPLE 1

Example 1 was prepared for purposes of demonstrating a desirable level 2 liquid and microorganism barrier could be achieved according to ANSI AAMI PB70. Biodegradability tests were not performed for Example 1.

A single layer sheet was manufactured on a Fourdrinier paper machine in the following manner: 34 percent bleached softwood cellulose fibers and a remainder consisting of 64 percent of natural fibers consisting of 20% of viscose fibers from Kelheim FibersGmbH (Viscose rayon DANUFIL® KS 1.7dTex) completed up to 64% by bleached softwood woodpulp such as eucalyptus and 2 percent of a binder and other compounds whose biodegradability is unknown were suspended in an aqueous medium. The natural fibers inherently included cellulose, but this cellulose is not included in the 34 percent by dry weight bleached softwood cellulose fibers. The binder and other compounds are selected among alkyl keten dimer, starch, and wet strength agent such as epychlorohydrine derivatives. The suspension was dewatered on the wire of the paper machine in order to form the sheet. The sheet was dried at around 120 degrees C. and the resulting sheet had a grammage of approximately 60 g/m².

The sheet was creped on-line to effect a softening of the sheet. The on-line creping was performed, resulting in conformability values of approximately 110 in the machine direction

The formed sheet was then tested to determine its characteristics with regard liquid and microorganism barrier, conformability, among other things.

### EXAMPLE 2

A single layer sheet was manufactured on a Fourdrinier paper machine in the following manner: 21 percent by dry weight cellulose fibers, having a length of 1.07 mm measured on Kajaani fiber length analyzer after 15 minutes of disintegration, were suspended in an aqueous medium with a remainder consisting of 76 percent by dry weight natural fibers having a length of 1.32 mm measured on Kajaani fiber length analyzer after 15 minutes of disintegration and synthetic acrylic binder and compounds with unknown biodegradability in a concentration of 3 percent by dry weight. The origin of the natural and cellulosic fibers was softwood. The natural fibers inherently included cellulose, but this cellulose is not included in the 21 percent by dry weight cellulose fibers. The suspension was dewatered on the wire of the paper machine in order to form the sheet. The sheet was dried at around 120 degrees C. and the resulting sheet had a grammage of approximately 75 g/m².

The sheet was then micro-creped to effect a softening of the sheet. The micro-creping was performed resulting in a conformability value of approximately 77 in the machine direction.

The formed sheet was then tested to determine its characteristics with regard, liquid and microorganism barrier, conformability, and biodegradability. among other things.

### EXAMPLE 3

A single layer sheet was manufactured on a Fourdrinier paper machine in the following manner: 16 percent cellulose fibers were suspended in an aqueous medium with a remainder consisting of 82 percent of natural fibers (bamboo fibers from OG Corporation) and 2 percent of a binder and other compounds whose biodegradability is unknown. The natural fibers inherently included cellulose, but this cellulose is not included in the 16 percent by dry weight cellulose fibers. The suspension was dewatered on the wire of the paper machine in order to form the sheet. The sheet was dried at around 120 degrees C. and the resulting sheet had a grammage of approximately 85 g/m².

The sheet was then embossed to effect a softening of the sheet. The embossing was performed resulting in a conformability value of approximately 90 in the machine direction.

The formed sheet was then tested to determine its characteristics with regard to, liquid and microorganism barrier, conformability, and biodegradability among other things.

### EXAMPLE 4

In a preferred mode, a single layer sheet was manufactured on a Fourdrinier paper machine in the following manner: 29 percent by dry weight cellulose fibers were suspended in an aqueous medium with a remainder consisting of 69 percent by dry weight natural fibers and 2 percent of binders and compounds whose biodegradability is unknown. Preferably, the cellulose fibers are obtained from hardwood wood pulp such as eucalyptus. The natural fibers are preferably obtained from unbleached hardwood woodpulp such as, for example, those sold by Canfor, or Arauco Companies. The natural fibers inherently included cellulose, but this cellulose is not included in the 29 percent by dry weight cellulose fibers. The suspension was dewatered on the wire of the paper machine in order to form the sheet. The sheet was dried at around 120 degrees C. and the resulting sheet had a grammage of approximately 75 g/m².

The sheet was then micro-creped on a microcreper to effect a softening of the sheet. The micro-creping was performed resulting in a conformability value of approximately 71 in the machine direction.

The formed sheet was then tested to determine its characteristics with regard to liquid and microorganism barrier, conformability, and biodegradability, among other things.

### EXAMPLE 5

A single layer sheet is manufactured on a Fourdrinier paper machine in the following manner: 16 percent by dry weight cellulose fibers were suspended in an aqueous medium with a remainder consisting of 78 percent by dry weight natural fibers, five percent of microcrystalline cellulose such as ARBOCEL® or CARBOCEL® products provided respectively by J Rettemenmaier & Sons GmbH and by Lamberti Company and 1 percent of binders and compounds of unknown biodegradability. The cellulose fibers are obtained from harwood woodpulp and natural fibers are obtained from unbleached harwood woodpulp. The natural fibers inherently included cellulose, but this cellulose is not included in the 16 percent by dry weight cellulose fibers. The suspension was dewatered on the wire of the paper machine in order to form the sheet. The sheet was dried at around 120 degrees C. and the resulting sheet had a grammage of approximately 75.5 g/m².

The sheet was then micro-creped to effect a softening of the sheet. The micro-creping was performed resulting in a conformability value of approximately 72 in the machine direction.

The formed sheet was then tested to determine its characteristics with regard to, liquid and microorganism barrier, conformability, and biodegradability.

TABLE 3

| Characteristic (units) | Standard | Ex. 1 | Ex. 2 | Ex. 3 | Ex. 4 | Ex. 5 |
|---|---|---|---|---|---|---|
| Grammage (g/m²) | ISO 536 (August | 60 | 75 | 82 | 75 | 75.5 |
| | 1996 version) | | | | | |
| Drape MD (mm) | EN 868-2 App. D | 110 | 77 | 90 | 71 | 72 |
| Aerobic Biod. (%\|days) | ISO 14851 | nd | 100 \| 28 | 85 \| 42 | 94.1 \| 36 | 92 \| 32 |
| Spray Impact Barrier (g) | AATCC42 | 0.06 | 4 | 0.03 | <1 g | 0.06 |
| Hydrohead (cm) | AATCC127 | 40 | n.a. | 60 | 50 | 55 |
| AAMI barrier Level | PB70 | 2 | 1 | 3 | 2 | 3 |
| Linting (log₁₀) | ISO 9073-10 | 4.4 | 4.5 | 4.4 | 4.2 | 4 |

Thus, based on embodiments of the present disclosure, a biodegradable monolayer medical barrier having desirable properties may be formed. As shown at Table 3, such exemplary embodiments may meet levels, 1, 2, and/or 3 barrier performance as set forth by AAMI, while also having desirable drape values, biodegradability, breathability, and strength among other things. Moreover, because a large portion, and in some cases all, of the fibers used are natural, monolayer medical barriers prepared according to embodiments of the present disclosure may be void of any eco-toxic substances, and more particularly may be PFOA free.

Throughout the description, including the claims, the term “comprising a” should be understood as being synonymous with “comprising at least one” unless otherwise stated. In addition, any range set forth in the description, including the claims should be understood as including its end value(s) unless otherwise stated. Specific measurement values for described elements should be understood to be within generally accepted manufacturing or industry tolerances, and any use of the terms substantially and/or approximately should be understood to mean falling within such generally accepted tolerances. Component ratios throughout the disclosure shall be understood to be by dry weight unless otherwise specified.

Where any standards of national, international, or other standards body are referenced (e.g., ISO, AAMI, etc.), such references are intended to refer to the standard as defined by the national or international standards body as of the priority date of the present specification. Any subsequent substantive changes to such standards are not intended to modify the scope and/or definitions of the present disclosure and/or claims.

Although the present disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

1. A biodegradable monolayer medical material, comprising at least 15 percent cellulose fibers by dry weight, with a remainder consisting of at least 60 percent by dry weight of natural fibers,
the monolayer medical material providing a barrier to liquids and/or microorganisms meeting at least level 1 performance based on a standard defined by AAMI PB70 (2008 version),
wherein at least a portion of the monolayer medical material comprises a softening process pattern configured to soften the monolayer medical barrier material.

2. The biodegradable monolayer medical material according to claim 1, wherein the barrier is configured to meet at least level 2 performance requirements based on the standard defined by AAMI PB70 (2008 version).

3. The biodegradable monolayer medical material according to claim 1, wherein the barrier is configured to meet at least level 3 performance requirements based on the standard defined by AAMI PB70 (2008 version).

4. The biodegradable monolayer medical material according to claim 1, wherein the softening process pattern is selected from a group consisting of a creped pattern, a micro-creped pattern, and an embossing pattern.

5. The biodegradable monolayer material according to claim 1, wherein the softening process pattern is formed on-line.

6. The biodegradable monolayer material according to claim 1, wherein the softening process pattern is formed offline.

7. The biodegradable monolayer medical material according to claim 1, wherein the softening process pattern results in a machine direction drape value lower than 200 mm.

8. The biodegradable monolayer medical material according to claim 7, wherein the softening process pattern results in a machine direction drape value lower than about 120 mm.

9. The biodegradable monolayer medical material according to claim 1, wherein the cellulose comprises at least one of bleached cellulose, semi-bleached cellulose, and unbleached cellulose.

10. The biodegradable monolayer medical material according to claim 9, wherein the cellulose comprises virgin and/or recycled cellulose.

11. The biodegradable monolayer medical material according to claim 1, comprising less than 20 percent microcrystalline cellulose.

12. The biodegradable monolayer medical material according to claim 11 comprising less than 10 percent microcrystalline cellulose.

13. The biodegradable monolayer medical material according to claim 1, wherein the natural fibers consisting of at least one of bleached wood pulp, semi-bleached wood pulp, unbleached wood pulp, cotton fibers, abaca, straw, bamboo, viscose, hemp, jute, sisal, and esparto (alfa).

14. The biodegradable monolayer medical material according to claim 1, wherein the natural fibers are unbleached cellulose fibers.

15. The biodegradable monolayer medical material according to claim 1, comprising less than 20 percent by dry weight of at least one of a synthetic compound, a compound with unknown biodegradability, and a non-biodegradable compound.

16. The biodegradable monolayer medical material according to claim 15, wherein the synthetic compound and /or the non-biodegradable compound comprises a binder.

17. The biodegradable monolayer medical material according to claim 1, having a relative aerobic biodegradability of at least 80 percent after 180 days.

18. The biodegradable monolayer medical material according to claim 17, having a relative aerobic biodegradability of more than 90% after 40 days.

19. The biodegradable monolayer medical material according to claim 1, wherein the monolayer medical material is void of any eco-toxic substances in a concentration greater than 200 parts per million and perfluoro octanoic acid free.

20. The biodegradable monolayer medical material according to claim 1, having a grammage between 25 g/m² and 200 g/m².

21. The biodegradable monolayer medical material according to claim 1 having a linting value of less than 10.

22. The biodegradable monolayer medical material according to claim 1 having a linting value of less than about 5.0.

23. The biodegradable monolayer medical material according to claim 1 wherein the cellulose fibers have an average length of between about 0.1 millimeter and about 30 millimeters.

24. The biodegradable monolayer medical material according to claim 23 wherein the cellulose fibers have an average length of between about 1 to 15 millimeters.

25. A sterilized surgical drape comprising the biodegradable monolayer medical material of claim 1.

26. The sterilized surgical drape according to claim 25, wherein a hole is provided in a predetermined location.

27. A sterile field comprising the biodegradable monolayer medical material of claim 1.

28. A sterilized gown comprising the biodegradable monolayer medical material of claim 1.

29. A sterilized sterile barrier system based on a standard defined by ISO 11607 (version of August 2009) comprising the biodegradable monolayer medical material of claim 1.

30. The biodegradable monolayer medical material according to claim 1 packed in or constituting a component of a sterilized sterile barrier system based on a standard defined by ISO 11607 (version of August 2009).

31. A method for manufacturing a biodegradable monolayer medical material, comprising:
preparing a furnish comprising at least 15 percent cellulose fibers by dry weight, with a remainder consisting of at least 60 percent by dry weight of natural fibers;
forming a sheet from the furnish in a wet-laid process;
drying the sheet; and
forming a pattern on and/or within the sheet via a softening process configured to result in a softening of the sheet and a barrier performance of the sheet to at least level 1 based on a standard defined by AAMI PB70 (2008 version).

32. The method according to claim 31, wherein the softening process is performed on-line and/or off-line and wherein the softening process is selected from at least one of creping, micro-creping and/or an embossing.

33. The method according to claim 31, wherein the softening process is configured to result in a machine direction drape value of between less than 200mm.

34. The method according to claim 33, wherein the softening process is configured to result in a machine direction drape value of less than 120 mm.

35. The method according to claim 31, wherein the wet-laid process is selected from one of a fourdrinier process, an inclined wire process, and a mold table process.
